(19) United States
(12) Patent Application Publication
(10) Pub. No.: US 2009/0071496 A1
(43) Pub. Date: Mar. 19, 2009

(54) Kit and Method for Hair Treatment Applications

(76) Inventors: Robert Wayne Glenn, JR., Ohio, OH (US); Paul James Smith, Twickenham (GB)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/133,575

(22) Filed: Jun. 5, 2008

(30) Foreign Application Priority Data
Jun. 6, 2007 (EP) 07109742.2

(51) Int. Cl. A61K 8/00; A61K 8/18; A61Q 5/06; A61Q 5/00; A45D 19/00

(52) U.S. Cl. 132/208; 424/70.1; 8/405; 424/70.24; 132/270

(57) ABSTRACT

A kit and a method to deliver at least two predetermined amounts of hair treatment composition is disclosed. The kit comprises individually packaged hair treatment compositions, at least one dispensing means and one or more hair treatment application devices. With the kit according to the present invention, it is possible to deliver substantially identical predetermined amount of hair treatment composition or a first and a second predetermined amounts of hair treatment composition which amounts have a ratio of from 4:1 to 25:1.

### FIELD OF THE INVENTION

The present invention relates to a kit and to a method to treat hair which allows for precise and non-messy targeted delivery to a hair strand, preferably to a bundle of hair strands, of at least two predetermined amounts of hair treatment composition within a hair treatment application device.

### BACKGROUND OF THE INVENTION

Hair treatment application devices are known in the art of hair cosmetics and may facilitate the application of hair treatment compositions to the hair. Depending on the desired treatment, said device is engineered and designed to perform the application of the cosmetic composition. An example for hair highlighting is the cap and hook system, which allows the selection and separation of single hair bundles for receiving a highlighting composition. Another example is a curler for selecting hair bundles to be permanently waved. Once these devices have been positioned onto the head, they do not require the user to have any special skills for applying the hair treatment composition to the selected hair strands.

These days however, the retail market proliferates with other types of hair treatment application devices. These devices generally comprise one or more areas, cavities or the like in which the hair treatment composition is accommodated. Loading of the hair treatment composition into the device may be accomplished by simply dipping the device in a bowl where the hair treatment composition has been previously prepared or by employing dispensing means. Once the device is ready to be used, it is contacted with the hair to be treated to deliver the composition to the hair.

Irrespective of how the composition is loaded, the user must take into account how much hair treatment composition is to be loaded to perform the desired treatment in a clean and tidy fashion. Usually these types of devices are designed to have a size ergonomically acceptable to be held by the user's hand. Given their size, after the first application these devices may need to be reloaded due to their limited capacities and thus, they may undergo multiple refilling steps within a single hair treatment experience.

Hair treatment compositions for hair cosmetics, especially those that perform highlighting and oxidative dyeing are very viscous as they need to stay in place where they are applied on the hair for a time sufficient to perform bleaching and/or oxidative dyeing. Due to their viscous nature, the hair treatment compositions adhere and can remain on the device's surfaces. Therefore, only a portion of what is delivered into the device is in fact applied to the hair. Thus, after the first loading of an empty and clean device, the user is confronted with having to refill the device again.

On the one hand if the user over-fills the device, the hair treatment composition may ooze or leak from the device during the second application of hair treatment composition to the hair causing messiness. Leaking and oozing are unwanted not only for cleanliness purposes. To achieve the expected end result it is necessary for the composition to be precisely applied where desired. Even a small amount of composition at the edge of the device may still provide unexpected results as it could easily transfer from a hair strand, preferably to a bundle of hair strands, to unselected ones, especially at the root-line. As a result, an unanticipated and undesired overall final appearance may occur. It should also be considered for toxicological and safety reasons that if unexpectedly the hair treatment composition is delivered at the root-line, it may also be transferred to the scalp where it may cause unnecessary irritation.

On the other hand, if the user under-fills the device, the hair treatment composition present within the device may be insufficient for performing a complete and even application on a single hair bundle. As a result, unless the user has very short hair, to accomplish a single root to tip application the user has to stop, refill and then start the application again by reapplying at mid length.

Thus, it still remains to be solved a way to provide controlled delivery of hair treatment composition to a hair treatment application device to enable users to perform multiple applications of hair treatment composition within a single cosmetic treatment in a clean and non-messy fashion.

The art teaches different approaches to address this problem either with increasingly more sophisticated devices such as described in EP1264559 or with devices incorporating mechanical/electrical power in combination with a large product storage area, such as in JP2002034636 and DE4041742. Such approaches present complexity in manufacture and costs.

It has now been surprisingly found that a kit and method of treatment, as defined herein after, can significantly improve the delivery of hair treatment composition to hair treatment application devices and can reduce messiness during the application of hair treatment composition even after multiple refilling steps.

### SUMMARY OF THE INVENTION

According to the invention, a hair treatment kit is provided, said kit comprising:

a. one or more individually packaged hair treatment compositions;

b. one or more hair treatment application devices; and

c. at least one dispensing means to deliver at least two predetermined amounts of said one or more individually packaged hair treatment compositions to said one or more hair treatment application devices; and

wherein said at least two predetermined amounts comprise a first predetermined amount and a second predetermined amount of hair treatment composition.

Furthermore, a method to treat hair by means of a kit according to the invention is also described.

### BRIEF DESCRIPTION OF THE DRAWINGS

FIG. 1A is a frontal view of the bottom plate (10) of a hair treatment application device according to the kit of the present invention. Said bottom plate (10) has a first ring of non-woven (50) on the internal surface (101). Part of the connection (30) is shown.

FIG. 1B is a frontal view of the top plate (20) of a hair treatment application device according to the kit of the present invention. Said top plate (20) has an internal surface (201) with a second ring (51) and a disk (52) of now-woven attached thereon. Two stoppers (40; 41) extend upwardly from the internal surface of the top plate (20). Part of the connection (30) is shown.

FIG. 1C is a perspective view of the hair treatment applicator device with the bottom plate (10), the connection (30) and the top plate (20) as shown in FIGS. 1A and 1B.

FIG. 2 shows the average on-hair dosage, the average evenness and the messiness of a hair treatment application device loaded with less, equal or more of its average available loading volume.

FIG. 3 shows the effect of subsequent loadings of the hair treatment application device with amounts of hair treatment composition substantially identical to the average available loading volume.

FIGS. 4A, 4B, 4C and 4D show the average on-hair dosage, average evenness and messiness achieved when the same hair treatment application device is loaded with a first predetermined amount of hair treatment composition and subsequently to the application of at least part of that first predetermined amount of hair treatment composition to the hair, with a second predetermined amount of hair treatment composition. The ratio between said first and second amount of hair treatment composition is within the ranges claimed in the present invention.

### DETAILED DESCRIPTION OF THE INVENTION

The present invention is characterized by the combination of the features described herein and how they jointly contribute when combined in a kit all together to the solution of the above described technical problem.

For the purpose of this invention, the term “hair strand” means a single keratinous fibre, and the term “bundle of hair strands” means a plurality of hair strands according to the meaning given herein.

For the purpose of this invention, the term hair refers to both living hair i.e. on a living body and to non-living hair i.e. in a wig, hairpiece or other aggregation of non-living keratinous fibre. Mammalian, preferably human hair is intended.

As used herein, the terms apply, accommodate, allocate, coat, load, absorb, adsorb and adhere are all synonyms referring to delivering the hair treatment composition into the hair treatment application device according to the invention.

Within the scope of the present invention the term “substantially identical” means that the amounts of hair treatment composition delivered are virtually identical although as for any dispensing means a variation from amount to amount of about −5% to about +5% is considered acceptable.

While the specification concludes with claims, which particularly point out and distinctly claim the invention, it is believed the present invention will be better understood from the following description.

The kit according to the present invention comprises at least one dispensing means to deliver at least two predetermined amounts of hair treatment composition to a hair treatment application device. The dispensing means according to the present invention may further deliver to said hair treatment application device at least three, preferably at least four, more preferably at least five, even more preferably at least ten predetermined amounts of hair treatment composition.

Each of said predetermined amounts of hair treatment composition comprises an amount of hair treatment composition from 0.1 to 15.0 grams, preferably from 0.2 to 12.0 grams, more preferably from 0.5 to 10 grams of hair treatment composition.

Said at least two predetermined amounts of hair treatment composition comprise a first and a second predetermined amounts of hair treatment composition which are delivered to at least one or more hair treatment application devices. Said first predetermined amount of hair treatment composition is delivered to said hair treatment application device before delivering said second predetermined amount of hair treatment composition.

When said kit comprises more than one hair treatment application device, preferably at least two, more preferably at least three, even more preferably at least ten, even more preferably still at least twenty hair treatment application devices, then the ratio between said first and second predetermined amount of hair treatment composition is about 1:1. Preferably, said hair treatment application devices are substantially identical. Each of said hair treatment application device is to receive one predetermined amount of hair treatment composition.

The dispensing means comprised within the kit may deliver at least two substantially identical predetermined amounts of hair treatment composition; in addition said dispensing means delivers at least three, preferably four, more preferably at least five, even more preferably at least ten predetermined amounts of hair treatment composition, which are substantially identical to said first predetermined amount of hair treatment composition.

In one embodiment said kit preferably comprises a ratio of from 4:1 to 25:1 between said first and second predetermined amounts of hair treatment composition. More preferably said kit comprises one hair treatment application device to receive both said first and second predetermined amount of hair treatment composition and any subsequent amount of hair treatment composition. The dispensing means comprised within the kit may deliver at least a third amount of hair treatment composition which is substantially identical to said second predetermined amount of hair treatment composition, preferably at least a fourth, more preferably at least a fifth, even more preferably at least a tenth predetermined amount of hair treatment composition.

It is also understood that the difference in the amount of the predetermined amounts of hair treatment composition can be obtained through instructions and the use of a single predetermined amount dispenser. In such an embodiment the consumer could be directed via diagrams or written text to use the dispenser a certain number of times for the initial loading and then another number of times for the subsequent loading. For example using the dispenser four times for the initial loading and only once for subsequent loading to produce a ratio between said first and second predetermined amounts of hair treatment composition of about 4:1.

### 1. Hair Treatment Application Devices

The retail market proliferates with hair treatment application devices for applying hair treatment composition to the hair. Within the scope of the present invention, a hair treatment application device includes any device which comprises a bottom portion where the hair treatment composition may be delivered and a top portion which can be brought into a juxtaposed relationship to the bottom portion when the hair treatment application device is in use to treat the hair. Thus, combs, brushes and so called wand-like tools are excluded from this definition. Examples of hair treatment application devices according to the kit of the present invention include, but are not limited to, a single piece of paper or card or foil which is folded roughly in half to create bottom and top portions. The hair treatment application device can have additional edges either shaped from the paper or card or foil or made from a different materials adhered to the perimeter to help to contain the hair treatment composition within the hair treatment application device. Other examples of hair treatment application devices which can be comprised within the kit according to the present invention comprises two hinged plates, wherein one of said plate comprises a concaved plate in combination with either a flat, concave, convex or irregular shaped second plate. Additional materials and/or means may be incorporated onto the device to contain the hair treatment composition. These materials and/or means include, but are not limited to, gasket material, preferably at the edge one or both plates, bags for comprising one or more liquid composition, fibrous and/or porous material, bristles and teeth. Hair treatment application devices as described in GB2242357, U.S. Pat. No. 7,025,069, U.S. Pat. No. 3,030,968, U.S. Pat. No. 6,062,231 and US2004/0182408 may be used within the kit according to the present invention.

In particular, the kit according to the present invention comprises at least one hair treatment application device which comprises a bottom portion, preferably a bottom plate where the hair treatment composition may be delivered and a top portion, preferably a top plate, which can be brought into juxtaposed relationship to said bottom portion. Each of said bottom and top plates comprise an internal and external surface and said bottom and top plates are hinged so that when said top plate is brought into a juxtaposed relationship to said bottom plate, said internal surfaces are in an opposed relationship. Even more preferably, said bottom plate comprises a cavity for accommodating the hair treatment composition.

Preferably, said bottom and top plate are of ergonomic size. The shape of the plates may vary. Rectangular, square, circular, elliptical or oblong shapes may be useful as they are easy to manufacture but other shapes, particularly those that are easily recognised by the consumers may be used. Each plate may be independently flat or curved. Each plate or at least a portion thereof may comprise one or more cavities, preferably one or more concave cavities or one or more V-shaped grooves, U-shaped grooves or combinations thereof. Said cavities or grooves may be independently located on the internal or external surface of either or both plates. Each plate may be of the same or different size and shape. Each plate may be independently manufactured from any known material or combination of materials capable of supporting a hair treatment composition.

Said bottom and top plates may be brought into juxtaposed relationship by pivoting said top and bottom plate about said connection. Said connection allows said top and bottom plate to be brought into a substantially co-planar juxtaposed relationship, so that the minimal distance between said internal surfaces of said top and bottom plate is from about 0.1 cm to about 1.0 cm.

Preferably said connection is a hinge; even more preferably said hinge has spring back properties.

### 2. Method to Measure the Average Available Loading Volume of a Hair Treatment Application Device

The following method may be used to measure the amount of hair treatment composition that can be delivered to a hair treatment application device as described above.

The weight of the hair treatment application device is recorded and the bottom portion of the hair treatment application device, into which the hair treatment composition is to be delivered, is centrally secured onto the fixed circular compression plate (70 mm radius) of an Instron 5564. The square surface of an acrylate column of 1 cm×1 cm×5 cm (width×depth×length) is attached with 3M™ double sided tape to the moveable circular Instron compression plate (70 mm radius). The positioning of the column is such that its length extends downwards towards the designated top portion of the hair treatment application device, which upon compression would enable the hair treatment application device to be in a state as that used during application. The Instron 5564 configured with a 100 N load cell is operated in a compressive extension mode. Upon achieving a 0.01 N preload at a rate of 10 mm/min, the force and extension meters were zeroed and the moveable compression plate further lowered at a rate of 10 mm/min until a 30 N force is achieved whereupon the compressive extension was held for 30 seconds and then recorded. This determines the plate separation that the hair treatment application device would have while in use. The moveable Instron 5564 compressive plate is subsequently raised so that the acrylate column is no longer in contact with the device.

The device is removed from the Instron 5564 and loaded with a Carbopol™ 956 (available from Noveon) solution prepared according to table 1 below. The amount of Carbopol™ 956 solution loaded should exceed the amount which can be loaded in the device, the excess estimated visually. The viscosity of the Carbopol™ 956 solution is determined on a Brookfield DV-II+ viscometer with a S52 cone attachment. A sample of 0.5 ml of the Carbopol™ 956 solution is equilibrated at approximately 26.7° C. and 1 rpm for one minute prior to measurement, whereupon the readings are taken at 1 rpm. The density of the Carbopol™ 956 solution was determined on an Anton Paar Density Metter (DMA 35N).

TABLE 1
Carbopol ™ 956 solution for
measuring the available loading volume

| Ingredients | % w/w |
|---|---|
| De-ionized Water | q.s. to 100 |
| Carbopol ™ 956 | 3.47 |
| Average Viscosity | 35,300 cPs |
| Average Density | 1.012 g/cm³ |

The device loaded with Carbopol™ 956 solution was weighed and re-attached to the fixed Instron compressive plate as described above. The Instron 5564 configured with a 100 N load cell was again operated in a compressive extension mode. Upon achieving a 0.01 N preload at a rate of 10 mm/min, the force and extension meters were zeroed and the moveable compression plate further lowered at a rate of 10 mm/min until an extension determined and recorded above and equivalent to the plate separation of the device in use was achieved. Any excess of Carbopol™ 956 solution not accommodated within the device in the state to treat the hair is displaced. This compressive extension was held for 60 seconds during which time any displaced Carbopol™ 956 solution coating the exterior of the device was carefully removed by wiping with two plies of laboratory paper towel. The moveable Instron 5564 compressive plate was subsequently raised so that the acrylate column was no longer in contact with the device. The device and any residual Carbopol™ 956 solution within the device were weighed and the total grams of Carbopol™ 956 solution accommodated within the average available loading volume was calculated according to the following equation (A), given that the density of the Carbopol™ 956 solution is about 1:

Carbopol™ 956 solution accommodated=Weight(_{DEVICE+SOLUTION)}−Weight(_{DEVICE)} (A)

The experiment was repeated three times and the average available loading volume in grams was stated to one decimal places.

### 3. Experimental Data

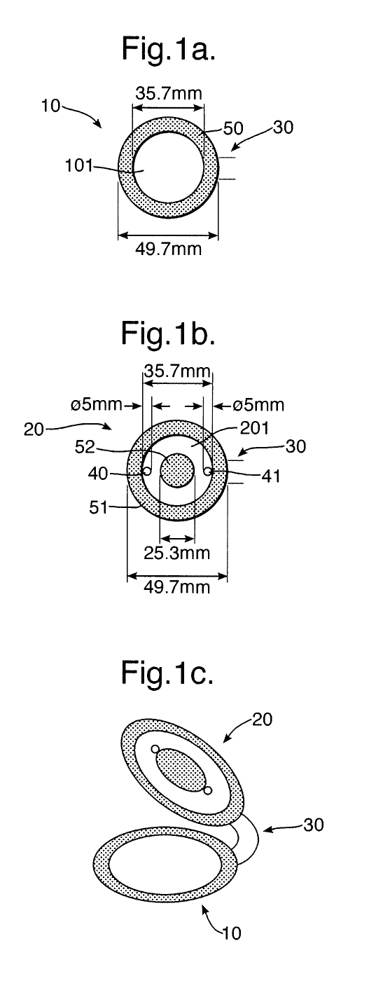

A hair treatment application device according to FIGS. 1a, 1b and 1c was arranged. The hair treatment application device comprises a bottom (10) plate articulated via a connection (30) to a top (20) plate. Each of said plates (10; 20) comprises an internal surface (101; 201). Two stoppers (40) are provided on the internal surface of said top plate (20). The plates (10; 20) are injection molded out of polypropylene together with the connection (30) and the two stoppers (40). A Libeltex Thermo-contact 01-766 DI-8 non-woven was cut into two rings (50, 51) and fixed with 3M™ 465 double sided tape on the internal surfaces (101; 201) of said plates (10; 20). Furthermore, a disk (52) of the same non-woven was attached internally to the ring on the internal surface (201) of said top plate (20). Utilizing the method to measure the available loading volume as described before, the volume of loading for this hair treatment application device was measured to be about 7.80 grams (about 100%, average available loading volume) being the bottom plate (10) the bottom portion and the top plate (20) the top portion.

With hair treatment application devices as described within the present invention, a user is faced with the major challenge of achieving firstly an application, secondly an even application and thirdly a non-messy even application and further being able to repeat the application on other hair strands as many times as needed. When all these conditions are met, the application is satisfactory and clean. Typically, indications in the form of instructions are given as to how to use the hair treatment application device. Nevertheless, it is very difficult for the user to achieve what is described in text and pictures. By having a dispensing means capable of delivering to the hair treatment application device predetermined amounts of hair treatment composition to provide a consistent satisfactory average on-hair dosage, average evenness and non-messiness is advantageous. The present inventors have found that to achieve average on-hair dosage, average evenness and non-messiness, the hair treatment application devices as described herein should be loaded to about 100% of the available loading volume, measured according to the method described above.

A solution of Carbopol™ 956 was prepared according to table 2 below. This solution comprises a pigment (Ultramarine Blue pigment and Titanium Dioxide) for the Carbopol™ 956 solution to be visually noticeable once applied to a tress of hair via the hair treatment application device of FIGS. 1A, 1B and 1C. The pigments are added to facilitate the visual detection of the Carbopol™ 956 solutions once they are deposited on the bundle of hair strands and when displaced outside the hair treatment application device. The ingredients were mixed and subsequently the viscosity was measured using the method described above. Four substantially identical hair treatment application devices as shown in FIGS. 1A, 1B and 1C were loaded each into the bottom plate (10) and each with a different amount of Carbopol™ 956 solution. The first device was loaded with an amount of Carbopol™ 956 solution corresponding to about 125% of the average available loading volume; the second, third, and fourth devices were loaded each with an amount of Carbopol™ 956 solution corresponding to about 100%, about 75%, or about 50%, respectively, of the average available loading volume (about 7.80 grams)

TABLE 2
Carbopol ™ 956 solution for comparative loadings
to about 100% of the average available loading volume

| Ingredients | % w/w |
|---|---|
| De-ionized Water | q.s. to 100 |
| Carbopol ™ 956 | 0.35 |
| Sodium Hydroxide (50% aq. Solution) | 1.49 |
| Ultramarine Blue pigment¹ | 0.05 |
| Titanium dioxide² | 0.27 |
| Average Viscosity | 35,300 cPs |
| Average Density | 1.038 g/cm³ |
| Average pH | 7.01 |

¹Ultramarine Blue pigment - Ultra Marine Blue 10-34-PC-3516 from Noveon, Inc.
²Titanium Dioxide - Hombitan FF Pharma from Sachtleben Chemie GmbH

A bundle of hair strands of 0.50 grams of hair, 30.5 cm long (Caucasian Light Brown—International Hair Imports and Products, Valhalla, N.Y.) was placed between the plates (10; 20) which were brought into juxtaposed relationship till the stoppers (40) stopped the compression. Each hair treatment application device was positioned at one end of the bundle of hair strands and then swiped twice along the length of the tress taking 3 seconds per swipe. The weight of the bundle of hair strands was recorded and the results calculated as grams of pigmented Carbopol™ 956 solution deposited per gram of hair.

The evenness and messiness of the coating were evaluated. The evenness of the coating was visually assessed for root-to-tip (along length of hair bundle); center-to-edge (across hair bundle width) and front-to-back (both sides of hair bundle) using a 1 to 5 rating scale (1 being poor evenness). Each rating was combined to provide an overall evenness value by averaging the root-to-tip; center-to-edge and front-back ratings. Messiness was visually assessed as pigmented Carbopol™ 956 solution displaced outside the hair treatment application device when the plates are brought into juxtaposed relationship.

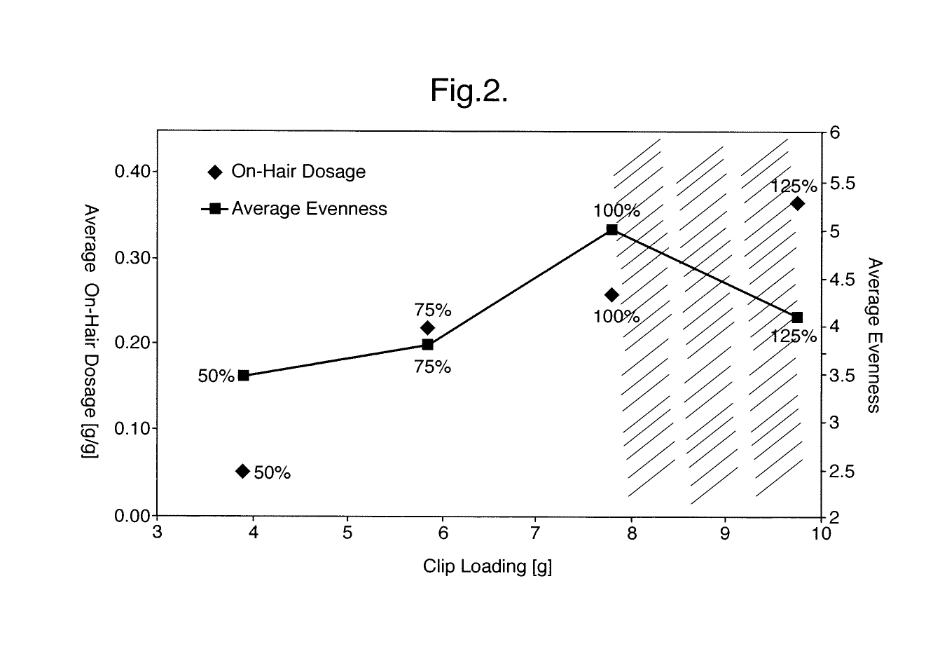

The results obtained are shown in FIG. 2. FIG. 2 depicts the average on-hair dosage (indicated by the symbol ♦) of pigmented Carbopol™ 956 solution deposited on the bundle of hair strands in grams per grams of hair and the average evenness (indicated by the symbol ▪) of the application versus the amount in grams of pigmented Carbopol™ 956 solution loaded into the hair treatment application device (clip loading on the X-axis of FIG. 2). When the hair treatment application device is loaded with an amount from about 75% to about 50% of the average available loading volume (about 7.80 grams), although the application onto the bundle of hair strands is not messy, the evenness of the application is poorer than that achieved when the device is loaded with about 100% of the average available loading volume (about 7.80 grams). In addition, the average on-hair dosage decreases as the loading volume decreases. When the hair treatment application device is loaded with about 125% of the average available loading volume, then the application becomes messy (indicated by the oblique lines) and although the average on-hair dosage increases, it translates in an overall poorly even application.

Thus, the kit according to the present invention preferably comprises a delivering means capable of delivering to a hair treatment application device a first predetermined amount of hair treatment composition which is substantially identical to about 100% of the average available loading volume measured according to the test method described herein.

Hair strand effects such as highlighting and oxidative dyeing are performed all over the head, or eventually in certain areas of the head, such as around the face to frame it with a lighter or different colour. One or more applications may be needed to complete the beauty experience.

To perform more applications, either a new hair treatment application device may be used, or the same device may be refilled.

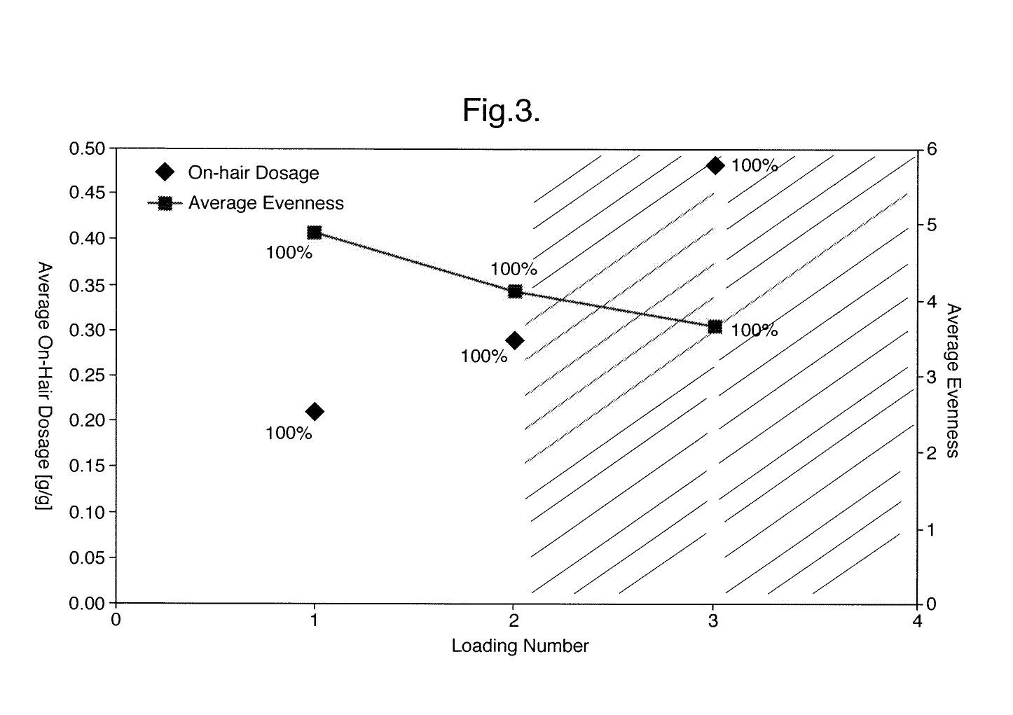

A hair treatment application device as described above was loaded with about 100% of the average available loading volume (about 7.80 grams) as a first predetermined amount of pigmented Carbopol™ 956 solution. A bundle of hair strands as described above was swiped twice. The weight of the bundle of hair strands after this first application was recorded as grams of pigmented Carbopol™ 956 solution deposited per gram of hair. The average evenness and messiness were also recorded as described above. The device was then reloaded with about 100% of the available loading volume as a second predetermined amount of hair treatment composition. A second bundle of hair strands was treated as before and the weight was recorded. The average evenness and the messiness of the application were recorded again. The step of re-loading and applying was repeated another time and the results are compared and shown in FIG. 3. After the first loading, the subsequent second and third loadings on the same hair treatment application device with about 100% of the average available loading volume are accompanied by messiness, as shown by the oblique lines, with most of the pigmented Carbopol™ 956 solution oozing and leaking out of the device while the swipe on hair occurs. Furthermore, although the average on-hair dosage increases, the average evenness of the application drops significantly.

As already mentioned above, it is the combination of maximal average on-hair dosage, maximal evenness and low or absent messiness which represents the result to be achieved for applications of hair treatment composition according to the invention.

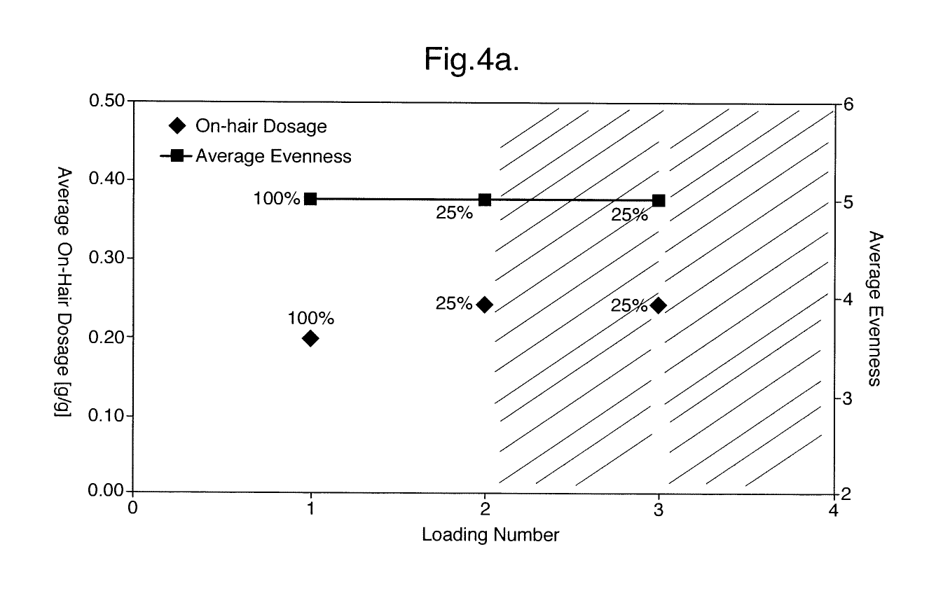

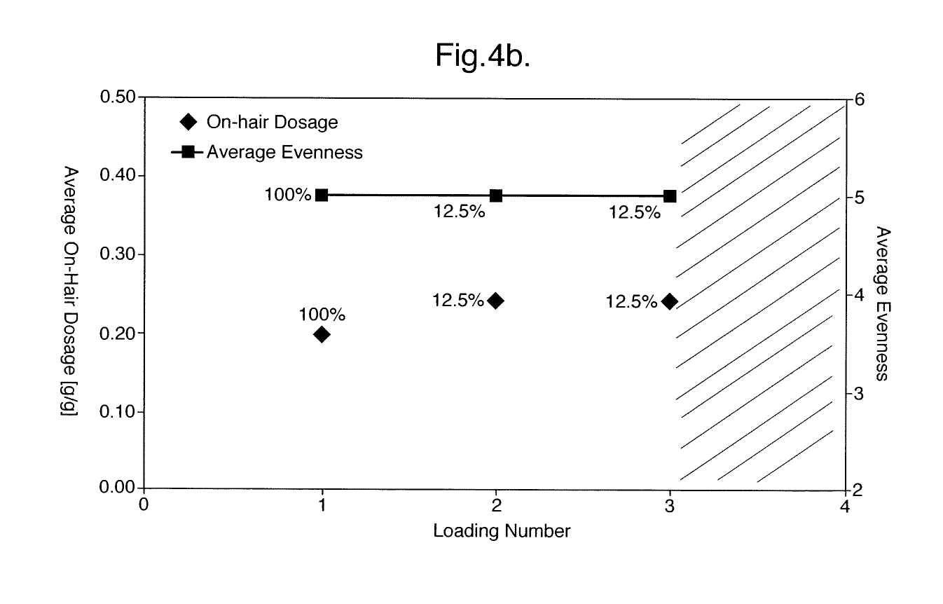

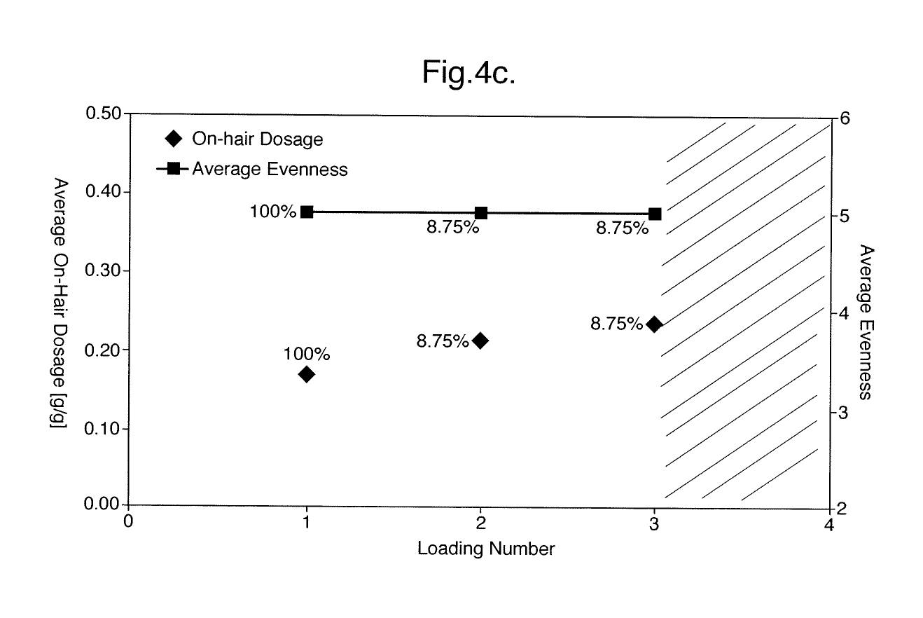

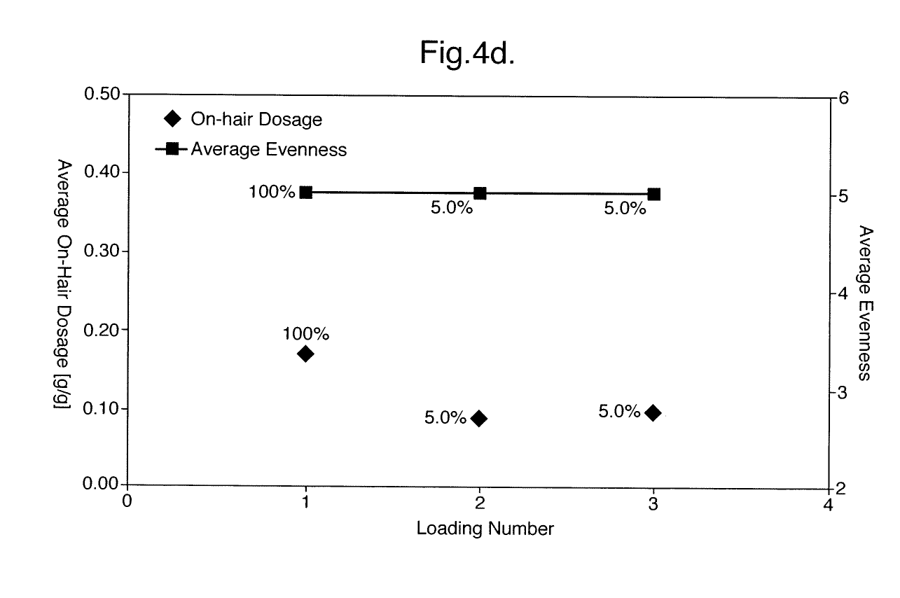

The present inventors have surprisingly found that the amount of hair treatment composition to be delivered as a second and further predetermined amount of hair treatment composition via a dispensing means to hair treatment application devices according to the invention is far from obvious for the consumer. The amount of hair treatment composition that needs to be delivered to the same hair treatment application device subsequently to the first predetermined amount of about 100% of the average available loading volume lays within a range of from about 25% to about 5% of the average available loading volume as show in FIGS. 4A, 4B, 4C and 4D.

FIGS. 4A, 4B, 4C and 4D all indicate the average on-hair dosage (indicated by the symbol ♦) of pigmented Carbopol™ 956 solution applied by a hair treatment application device as shown in FIGS. 1A, 1B and 1C when that device is loaded firstly with 100% of the average available loading volume and subsequently to the application of some pigmented Carbopol™ 956 solution to the hair is again loaded for a second and third time with amounts corresponding to about 25% (FIG. 4A), about 12.5% (FIG. 4B), about 8.75% (FIG. 4C) and about 5% (FIG. 4D) of the average available loading volume. For each of those second and third loadings, FIGS. 4A to 4D also show that the average evenness of the application (indicated by the symbol ▪) is constantly satisfactory (on a scale of from 1 to 5, being 5 the highest value).

In addition, FIG. 4A indicates that some messiness (indicated by the oblique lines) occurs when an amount of pigmented Carbopol™ 956 solution of about 25% of the average available loading volume is delivered as a second and third loading. As shown in FIGS. 4B and 4C, the messiness is even reduced to the third loading when the hair treatment application device is loaded with about 12.5% or about 8.75% of the average available loading volume as second and third loading.

Finally, as shown in FIG. 4D, no messiness is observed when the hair treatment application device is again loaded for a second and third time with an amount of pigmented Carbopol™ 956 solution corresponding to about 5% of the average available loading volume.

In conclusion, by loading the hair treatment application device with a first predetermined amount of hair treatment composition of about 100% of the average available loading volume and with a second predetermined amount of from about 8.75% to about 25% of the average available loading volume, the average evenness and average on-hair dosage achieved are satisfactory enough to compensate for some messiness. When the hair treatment application device is loaded with a second and third predetermined amount of hair treatment composition which is about 5% of the average available loading volume, no substantial leaking of the hair treatment composition from the hair treatment application device is observed although the average on-hair dosage after each re-fill does initially slightly decrease and then remains constant.

Thus, when the ratio between the first and the second predetermined amount of hair treatment composition is from about 4:1 to about 25:1, achieved the best combination of average evenness, average on-hair dosage and messiness are achieved.

### 4. Dispensing Means

Dispensing means within the meaning of the present invention are capable of metering at least two amounts of hair treatment composition so as to deliver it as predetermined amounts of hair treatment composition to the hair treatment application device. In one embodiment one or more hair treatment compositions are stored separately from one another. The hair treatment compositions are mixed to homogeneity in one or more containers to form a final composition, which is then delivered as first and second predetermined amounts of hair treatment composition into one or more hair treatment application devices. In another embodiment, only one type of hair treatment composition is delivered as first and second predetermined amount of hair treatment composition.

In one embodiment according to the present invention, a kit is provided comprising a hair treatment application device as described herein below; a first and second individually packaged hair treatment compositions, and a bowl to mix said first and second individually packaged hair treatment compositions. Within the kit a scoop is also provided as a dispensing means to deliver substantially identical predetermined amounts of hair treatment compositions. Said at least two individually packaged hair treatment compositions, which can both be liquid, cream or a mixture of liquid or cream and powders, are transferred into the bowl and mixed together to homogeneity to form a third hair treatment composition. The open curved surface of said scoop is dipped into the bowl to pick up a pre-determined amount of said third hair treatment composition. The amount of said third hair treatment composition in said scoop may be optionally levelled off using as reference the lateral walls of said scoop to obtain further accuracy in the predetermined amount of hair treatment composition. Said predetermined amount of hair treatment composition is then delivered into the hair treatment application device. The hair treatment composition is then applied to the hair. The steps of dip in, pick up, optionally level off, deliver, and finally application of the hair treatment composition to the hair, are repeated as many times as needed until the entire treatment is complete. When substantially identical predetermined amounts of hair treatment composition are delivered to said hair treatment application device, said kit preferably comprises at least two hair treatment application devices, preferably at least three, more preferably at least ten, even more preferably at least twenty hair treatment application devices. Said devices may be different or substantially identical, preferably substantially identical. Each of said hair treatment application devices is for receiving one of said substantially identical predetermined amounts of hair treatment composition.

In another embodiment according to the present invention said kit comprises one or more individually packaged hair treatment compositions, a hair treatment application device a bowl and a first and second scoop. Said first scoop is for delivering a first predetermined amount of hair treatment composition and said second scoop is for delivering a second predetermined amount of hair treatment composition to said hair treatment application device. The ratio of the amounts of hair treatment composition which can be delivered by said first and second scoops is comprised within the ratio described is claimed herein. The second scoop is also used to deliver any additional predetermined amount of hair treatment composition. Once said one or more individually packaged hair treatment compositions have been mixed within the bowl to homogeneity, the first scoop is dipped into the bowl to pick up a first predetermined amount of hair treatment composition. The latter may be optionally leveled off as described above to improve the accuracy of the predetermined amount. Said predetermined amount of hair treatment composition is delivered to said hair treatment application device. Said device is then contacted with the hair to apply at least a portion of said hair treatment composition. The application of the hair treatment composition to the hair may be repeated twice or even three times before refilling.

In another embodiment of the kit according to the present invention, said one or more scoops are replaced with one or more pipettes or syringes. A pipette and a syringe are both characterized by the presence of a cylindrical container. One of the extremities of said cylindrical container terminates with an orifice where the hair treatment composition may be delivered. The other extremity is, in a syringe, for receiving a plunger. The plunger may be pushed and pulled along inside the cylindrical container. In a pipette the other extremity is connected to a mean to provide vacuum. The vacuum may be provided manually using a bulb or mechanically by a piston-driven air displacement tool. In one example the cylindrical container of said syringe or pipette comprises gradation marks at specific intervals. The amount of hair treatment composition comprised between two consecutive gradation marks is a predetermined amount of hair treatment composition. One or more predetermined amounts of hair treatment composition are delivered to said hair treatment application device. The hair treatment composition is dispensed from the bowl via the syringe or pipette to the hair treatment application device. In another embodiment more than one pipette or syringe is provided as means to obtain different predetermined amounts of hair treatment composition.

In another embodiment within the scope of the present invention, said kit may comprise one or more individually packaged hair treatment compositions, one or more hair treatment application devices and a bottle. Said bottle is used to mix and dispense said first and second individually packaged hair treatment compositions. One or more of said individually packaged hair treatment compositions may be already stored within the bottle. Said bottle may be provided with graduation marks. The graduation marks are placed on the side wall of said bottle at intervals. The minimal distance between two consecutive gradation marks multiplied by the cross-sectional area normal to the longest axis of said bottle corresponds to a predetermined amount of hair treatment composition. Said predetermined amount may be delivered to said hair treatment application device by squeezing the walls of said bottle forcing the hair treatment composition to be released through the opening of said bottle or, if a nozzle is attached to that opening, through said nozzle.

In another embodiment of the present invention, the kit may comprise a dual sachet. Each compartment of said dual-sachet is separated one from the other by a physical frangible barrier and each compartment comprises a distinct hair treatment composition. Said barrier can be removed during mixing of said distinct hair treatment compositions. Once mixing is completed, the sachet is opened and the content is delivered to said hair treatment application device as a predetermined amount of hair treatment composition. If a different predetermined amount of hair treatment composition is required for the subsequent delivery, then the second and any subsequent dual-sachet may comprise an amount of hair treatment composition different from that of the first dual-sachet.

In a preferred embodiment of the kit according to the invention said at least one dispensing means is a lotion pump. The lotion pump is preferably attached to the neck of a bottle or of a tube. The lotion pump allows for precise and constant delivery of predetermined amounts of hair treatment composition. The lotion pump is attached to a tube to draw the hair treatment composition from the bottle and to a trigger lever to activate the lotion pump, wherein said trigger lever comprises a nozzle to discharge the hair treatment composition. Preferably, the lotion pump comprises a piston housed in a cylinder. The piston is connected to the trigger lever. Inside the cylinder there is a spring and when the trigger lever is pushed down, the piston compresses the spring and when the trigger lever is released, the spring pushes back the trigger lever to its original position. The down-stroke of the piston pushes out an amount of hair treatment composition comprised within the tube and the cylinder and the up stroke of the piston pulls up in the tube the hair treatment composition from the bottle. Each time that the piston is activated through the trigger level, substantially identical amounts of hair treatment composition are dispensed.

In another preferred embodiment of the kit the lotion pump may be provided with a mechanical stop that limits the amount of hair treatment composition which is delivered by the lotion pump at each down- and up-stroke of the trigger lever. Once a first predetermined amount of hair treatment composition is delivered, a band or strap of non-compressible material is engaged between the trigger lever and the spring so that the piston cannot be entirely stroked. As a result, the ratio between the first predetermined amount of hair treatment composition and this second amount of hair treatment composition is within the ranges claimed herein.

Preferred commercially available pumps are those provided by Rieke Packaging Systems (Leicester, England) in particular those sold commercially under the names of the Swift Doser, the Screw Collar Falcon, the Essential Dispenser, the Visor Dispensor, The Stork Dispenser, the Scorpio Dispenser, the RS3 Shipper Pump, the RS4 Shipper Pump, the RS5 Shipper Pump, and the RS10 Shipper Pump, and the HVDS series and HVDSL series of high viscosity dispensing systems. Other commercially available pumps include those sold by SeaquistPerfect Dispensing (Cary, Ill., USA) under the names of the Contemporary™ hoodless design, the Falcon™, the Colibri™, the SeaFlowr™, the Ibis™, the Flamingo™, the Cardinal™, and the Euroflow™.

According to the invention, said one or more hair treatment compositions are stored as individually packaged hair treatment compositions in one or more containers. Said containers comprise, but are not limited to, sachets, blisters, sachets and blisters having different compartments separated via frangible seals, syringes, tubes and bottles. When two or more compositions are present within the kit according to the present invention, said two or more hair treatment compositions may be stored in containers provided with multiple separated chambers. Preferably, said two or more hair treatment compositions are mixed before they are delivered to said one or more hair treatment application devices.

Multi-chambered packages may not only serve as housing for said one or more hair treatment compositions, they may also provide dispensing and/or mixing of said one or more hair treatment compositions. Suitable examples comprise but are not limited to, dual-syringes, dual-tubes, dual-chamber squeeze packaging, dual-chamber lotion pumps, dual chamber non-aerosol pump dispensers, dual chamber non-aerosol foamers, dual-chamber variable non-aerosol pumps, dual-chamber volumetric screw dispensers, dual-chamber airless pump dispensers, dual-chamber bag-in-can unit dose systems, dual-aerosol unit dose mousse systems, dual-compartmented aerosols, bag-in-bag in can aerosol and dual-piston and chamber systems, all available from various suppliers including Kloche, Hensen Packaging Concept GmbH (Verden, Germany), Adhesive packaging Specialists Inc. (Peabody, Mass.), Cebal (Norwalk, Conn.), Rexam (Purchase, N.Y.), Seaquist Closures (Mukwonago, Wis.), Perfect Valois UK Ltd. (Milton Keynes), Rexam Airspray Inc. (North Pompano Beach, Fla.), Hana Co. Ltd. (South Korea), Wiko (Morgantown, Pa.), Toyo Packaging Industries Ltd. (Singapore), Coster (South Elgin, Ill.), and Precision Valve Corporation (Yonkers, N.Y.).

In another preferred embodiment, a lotion pump is attached to a multi-chambered bottle. Each of said separate chambers provides for a separate container for each of said one or more hair treatment compositions and each chamber may be provided of its own lotion pump. In a more preferred embodiment, at least two hair treatment compositions are stored in a dual-chambered bottle. Said two hair treatment compositions comprise a first and a second hair treatment composition. Said dual-chambered bottle comprises a first chamber for said first hair treatment composition and a second chamber for said second hair treatment composition. Said first hair treatment composition preferably comprises an oxidizing agent whereas said second hair treatment composition comprises an alkalizing agent. By simultaneously activating said lotion pumps, said first and second hair treatment composition may be delivered to said hair treatment application device. In an even more preferred embodiment, said lotion pump is provided with only one trigger lever and between the nozzle of the trigger lever and the piston a static mixer is provided. Said static mixer may allow for the inter-mixing of said first and second hair treatment compositions to form a third hair treatment composition before said third hair treatment composition is delivered to said hair treatment application device as predetermined amount of hair treatment composition.

### 5. Hair Treatment Compositions and Additional Components

Examples of hair treatment compositions that may be delivered via said dispensing means to the hair treatment application device according to kit of the present invention are selected from the group consisting of styling compositions, dyeing compositions, highlighting compositions or combination thereof. In one embodiment of the present invention, said one or more individually packaged hair treatment compositions comprise a first individually packaged hair treatment composition and a second individually packaged hair treatment composition. When mixed said first and second individually packaged hair treatment compositions form a third hair treatment composition. In a preferred embodiment of the kit according to the present invention, said first individually packaged composition comprises an oxidizing agent and said second individually packaged composition comprises an alkalizing agent. Preferably, said oxidizing agent is hydrogen peroxide. More preferably, at least one of said first and/or second individually packaged hair treatment composition comprises a persulfate salt.

The kit according to the present invention may further comprise additional components such as means to select the hair strands, combs or brushes, gloves, caps with holes, tweezers, tongues, hooks or combination thereof.

The kit-of parts according to the present invention may further comprise instructions for using at least one component of the kit according to the invention. Preferably, said kit may comprise instructions for consumers indicating how to load and/or use components of said kit, said instructions being recorded in any type of media such as the package of the kit itself, paper material, compact disk or the dispensing means and/or the hair treatment application device itself.

### 6. Method of Use

According to the present invention a method to treat hair with the kit described herein is provided. Said one or more individually packaged hair treatment compositions are delivered to said hair treatment application device through said dispensing means as first predetermined amount of hair treatment composition. At least a portion of said first predetermined amount of hair treatment composition is applied with said hair treatment application device to the hair. Said one or more individually packaged hair treatment compositions are delivered through said dispensing means to said hair treatment application device or to another hair treatment application device as second predetermined amount of hair treatment composition. Said second predetermined amount of hair treatment composition is then applied to the hair. Any additional predetermined amount of hair treatment composition is delivered to a hair treatment application device and at least a portion thereof is then applied to the hair. The hair treatment composition is then left on the hair, and then optionally rinsed, optionally with a shampoo, and then dried.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments or the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair treatment kit comprising:
a. one or more individually packaged hair treatment compositions;
b. one or more hair treatment application devices; and
c. at least one dispensing means to deliver at least two predetermined amounts of said one or more individually packaged hair treatment compositions to said one or more hair treatment application devices; and
wherein said at least two predetermined amounts comprise a first predetermined amount and a second predetermined amount of hair treatment composition.

2. The kit according to claim 1, wherein each of said predetermined amounts of hair treatment compositions comprises an amount of hair treatment composition of from about 0.1 to about 15.0 grams.

3. The kit according to claim 1, wherein when the ratio between said first and second predetermined amount of hair treatment compositions is about 1:1, then said kit comprises more than one hair treatment application device.

4. The kit according to claim 3, wherein said at least one dispensing means further delivers to said hair treatment application device at least three predetermined amounts of hair treatment composition, which are substantially identical to said first predetermined amount of hair treatment composition.

5. The kit according to claim 1, wherein the ratio between said first and second predetermined amount of hair treatment compositions is from about 4:1 to about 25:1.

6. The kit according to claim 5, wherein said at least one dispensing means further delivers to said hair treatment application device at least a third predetermined amount of hair treatment composition which is substantially identical to said second amount of hair treatment composition.

7. The kit according to claim 1, wherein said one or more individually packaged hair treatment compositions are compositions selected from the group consisting of styling compositions, highlighting compositions, dyeing compositions and combinations thereof.

8. The kit according to claim 1, wherein said one or more individually packaged hair treatment compositions comprise at least a first individually packaged hair treatment composition and at least a second individually packaged hair treatment composition.

9. The kit according to claim 8, wherein said first and second individually packaged hair treatment compositions are mixed to form a third hair treatment composition, and wherein said first individually packaged hair treatment composition comprises an oxidizing agent and wherein said second individually packaged hair treatment composition comprises an alkalizing agent.

10. The kit according to claim 10, wherein at least one of said first or second individually packaged compositions comprises a persulfate salt.

11. The kit according to claim 1, wherein said at least one dispensing means is a lotion pump.

12. The kit according to claim 1 further comprising instructions for using at least one of the components of said kit.

13. The kit according to claim 1 wherein said at least one hair treatment application device comprises a bottom portion, where the hair treatment composition may be delivered, and a top portion, which can be brought into juxtaposed relationship to said bottom portion.

14. The kit according to claim 13, wherein each of said bottom and top plates comprise an internal and external surface and wherein said bottom and top plates are hinged so that when said top plate is brought into a juxtaposed relationship to said bottom plate, said internal surfaces are in an opposed relationship.

15. A method to treat the hair with a kit according to claim 1 comprising the steps of:
i. delivering through said dispensing means said one or more individually packaged hair treatment composition as said first predetermined amount of hair treatment composition to said hair treatment application device; and
ii. applying with said hair treatment application device at least a portion of said first predetermined amount of hair treatment composition to the hair.

16. The method according to claim 15, wherein said one or more individually packaged hair treatment composition are delivered as said second predetermined amount of hair treatment composition to said hair treatment application device or to another hair treatment application device through said dispensing means and wherein at least a portion of said second predetermined amount of hair treatment composition is applied to the hair with said hair treatment application device.
